(19) United States
(12) Patent Application Publication
(10) Pub. No.: US 2011/0190702 A1
(43) Pub. Date: Aug. 4, 2011

(54) MEDICATION DOSING DEVICE FOR ADMINISTERING A LIQUID MEDICATION

(76) Inventor: Michael Stumber, Korntal-Muenchingen (DE)

(21) Appl. No.: 12/734,757

(22) PCT Filed: Nov. 14, 2008
(86) PCT No.: PCT/EP2008/065609

§ 371 Date: Apr. 20, 2011

(30) Foreign Application Priority Data
Nov. 21, 2007 (DE) 102007055636.7

(51) Int. Cl. A61M 5/168; A61M 5/20

(52) U.S. Cl. 604/151; 604/131

(57) ABSTRACT

A medication dosing device for administering a liquid medication, having a housing, the housing having a medication reservoir, a medication dosing unit, a drive for conveying the medication, an administration needle, a power supply unit, and an electrical control system. The medication reservoir, the medication dosing unit, and the administration needle are jointly situated in a unit which may be inserted .into the housing and removed from the housing. The medication dosing device has the advantage that it may be easily manufactured and cost-effectively operated.

### FIELD OF THE INVENTION

The present invention relates to a medication dosing device for administering a liquid medication, having a housing, the housing having a medication reservoir, a medication dosing unit, a drive for conveying the medication, an administration needle, a power supply unit, and an electrical control system.

### BACKGROUND INFORMATION

Medication dosing devices have great importance in various fields of application, such as in the medical technology field, and are used, for example, for continuous administration of insulin to diabetes patients. Very small quantities of the medium must be individually dosed and administered.

Various implementations of medication dosing devices are known from the related art. Most of these medication dosing devices for continuous administration of insulin use insulin pumps to deliver an individual daily profile of insulin from a reservoir to the body. Requirements for sterility limit the usability, as well as the volume of the reservoir and also all components associated with the medication, to approximately three days.

A medication dosing device for administering liquid medications, in particular insulin, is described in DE 103 52 456, having a reservoir which is provided with a pump apparatus, and a supply line connected to the body of the patient. According to this design the pump apparatus and reservoir are provided in a common assembly, the reservoir preferably being replaceable.

This and other designs known from the related art are often bulky and expensive, which limits their widespread use. The reason is that precise and reliable designs are required. In addition, the sterility requirements described above limit the capability for reuse.

Other known designs use components such as micropumps for the purpose of miniaturization. Due to sterility requirements, the reservoir as well as the micropumps must be replaced, which makes the operation costly.

### SUMMARY OF THE INVENTION

In comparison to the related art, the medication dosing device according to the present invention has the advantage that it may be easily manufactured and cost-effectively operated.

This is achieved according to the present invention by using a medication dosing device for administering a liquid medication, having a housing, the housing having a medication reservoir, a medication dosing unit, a drive for conveying the medication, an administration needle, a power supply unit, and an electrical control system, in which the medication reservoir, the medication dosing unit, and the administration needle are jointly situated in a unit which may be inserted into and removed from the housing.

According to the present invention, a division is thus provided between reusable and replaceable components in such a way that only the components in direct contact with the medication and the patient are situated in the removable unit and have fluid connections between the individual components. In this way the components may be economically reused separately from the components situated in the removable unit. At the same time, the components which must meet sterility requirements are situated in the removable unit. As a result of the targeted division of the components, the manufacture of the medication dosing device is simplified, and a compact design of the overall unit may be achieved.

The removable unit may preferably be inserted into the medication dosing device by simple manipulation. A guide and/or a grid may be provided to facilitate the insertion. The dosing device may include a pump, a micropump, a valve, or a microvalve. The reservoir may be designed as a cartridge or a bag.

In the arrangement of the reservoir, the dosing device, and the administration needle in the removable unit, it is preferably provided that the removable unit is designed as a disposable article. The disposable article is sterilely packaged in the delivered state in order to meet sterility requirements. The disposable article is replaced as soon as the medication in the reservoir is used up or out of date, i.e., past its expiration date. When properly handled, the disposable article has better sterility characteristics than reusable reservoirs, dosing devices, or administration needles. Possible large-scale manufacture allows the disposable articles to be manufactured cost-effectively.

In one preferred refinement of the present invention, the removable unit designed as a disposable article also has a power supply unit. In this regard it is preferably provided that the disposable article is equipped with a battery. The battery provides the required amount of power for all electrical components of the medication dosing device during the useful life of the disposable article.

It is further preferred to design the power supply unit in the housing to be separate from the removable unit, and/or as a rechargeable battery. The removable unit may thus be manufactured in a more compact design. The rechargeable battery provides sufficient power for using multiple removable units and for the housing, separate from the removable unit. The costs for the removable unit and the medication dosing device are reduced due to the reusability of the rechargeable battery.

It is particularly preferred to design the drive for conveying the medication and/or the electrical control system in the housing to be separate from the removable unit. In this way, the size and the manufacturing costs for the removable unit are reduced. The drive for conveying the medication is particularly preferably designed using at least one piezoelectric actuator.

It is further preferred that a display device, a communication interface, at least one control element, and/or a needle insertion device be provided in the housing, separate from the removable unit. The display device displays the operating state of the medication dosing device, the state of the power supply unit, or the state of the rechargeable battery, for example. In addition, the display device may display the state of the reservoir, for example concerning the period of time the reservoir has been in operation or may remain in operation, or the quantity of medication that is still available in the reservoir, and the period of time this quantity will last.

With the aid of the communication interface it is possible, for example, to individually set the quantities of the medication to be dosed and administered at particular times. Data from the medication dosing device may also be read out with the aid of the communication interface. The read-out data are evaluated by software or by a physician, for example. The control element may be used, for example, to set the quantities of the medication to be dosed and/or to be administered at particular times. The control element is preferably designed as a switch or a pushbutton. The needle insertion device guides the administration needle in order to position the administration needle at an injection site as accurately as possible.

In one preferred specific embodiment at least one sensor is provided in the housing, separate from the removable unit. The sensor is provided for measuring values such as temperature, pressure, flow rate, blood parameters, or active substance levels. The values may be displayed on the above-described display device. It is also provided that the values may be transmitted via the communication interface for external evaluation. In one preferred specific embodiment the values are used as control parameters for the electrical control system.

In a further preferred specific embodiment the removable unit has at least one sensor component. Such a specific embodiment is particularly preferred when values are measurable via the sensor component which may be sampled only at the components which are in direct contact with the medication. Values such as temperature, pressure, flow rate, blood parameters, or active substance levels are measurable using the sensor component. The values may be displayed on the above-described display device. It may also be provided that the values may be transmitted via the communication interface for external evaluation. In one preferred specific embodiment the values are used as control parameters for the electrical control system.

According to a further feature of the present invention it is provided that the housing has a fastening device, separate from the removable unit, for affixing to the body of a patient. The fastening device is designed as a clip or a band, for example. The fastening device allows simple affixing to the body of the patient, and may be comfortably worn even for a fairly long period of time.

In one preferred specific embodiment the removable unit has a fastening device for affixing to the body of the patient. The fastening device is designed as a patch or adhesive connection, and may be used multiple times.

It is further preferred that the medication dosing device has a micropump made of silicon. It is particularly preferred that the micropump is designed as a micro diaphragm pump made of silicon glass or silicon plastic.

The medication reservoir of the previously described medication dosing device is preferably filled with a medication in the delivered state. In this way a supplier of the medication ensures that the medication is sterile and is optionally filled into the reservoir in a specified dosage. Colored marking and/or other labeling of the reservoir, for example a bar code or RFID, distinguishes various medications. The reservoir is labeled with information concerning the type of medication, required dosage, and/or shelf life.

The above-described medication dosing device is preferably used in the field of medical technology, for example for the previously discussed insulin administration, as well as for continuous local administration of pain medications or psychotropic drugs.

### BRIEF DESCRIPTION OF THE DRAWINGS

FIG. 1 shows a schematic illustration of a medication dosing device according to a first preferred exemplary embodiment of the present invention, with the important function blocks thereof.

FIG. 2 shows a schematic illustration of a medication dosing device according to a second preferred exemplary embodiment of the present invention.

FIG. 3 shows a schematic illustration of a medication dosing device according to a third preferred exemplary embodiment of the present invention.

### DETAILED DESCRIPTION

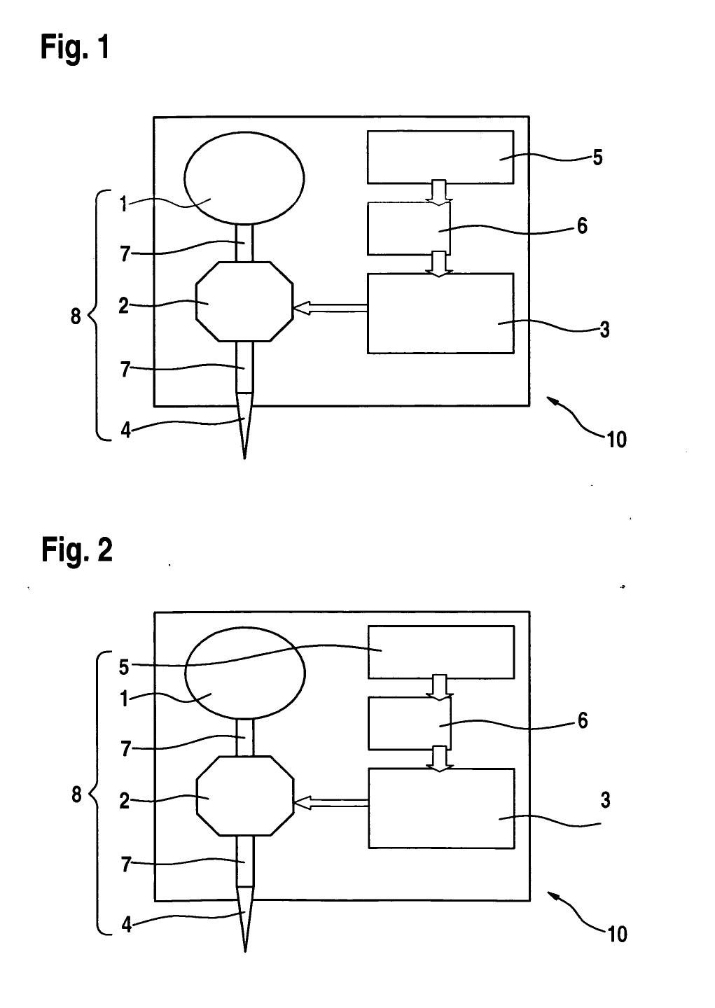

FIGS. 1 through 3 each show a schematic illustration of a medication dosing device according to a preferred exemplary embodiment of the present invention. These medication dosing devices are used for dosing very small quantities of a medication, and each have a medication reservoir 1, a medication dosing device 2, a drive 3 for conveying the medication, an administration needle 4, a power supply unit 5, and an electrical control system 6 as illustrated in FIG. 1, for example. According to the preferred exemplary embodiments of the present invention, these components of the medication dosing device are provided in a housing 10.

Medication reservoir 1, medication dosing device 2, and administration needle 4 have fluid connections 7 to one another. Medication reservoir 1 is connected via a first fluid connection 7 to medication dosing device 2, which is connected to administration needle 4 via a further fluid connection 7. Power supply unit 5 provides electrical control system 6 with power. Electrical control system 6 controls drive 3 for conveying medication, which drives medication dosing device 2. A medication may be filled into medication reservoir 1, and with the aid of medication dosing device 2, driven by drive 3 for conveying medication, is injected via administration needle 4.

As is apparent in FIG. 1, medication reservoir 1, medication dosing device 2, and administration needle 4 are jointly provided in a removable unit 8 which may inserted into and removed from housing 10. In the inserted state, removable unit 8 is a part of housing 10. All components of the medication dosing device in contact with the medication may be replaced by exchanging removable unit 8. All other components not in contact with the medication are reusable.

As is apparent in FIG. 2, according to a second preferred specific embodiment, power supply unit 5 is situated in removable unit 8. Exchanging removable unit 8 replaces not only the components in contact with the medication, such as medication reservoir 1, medication dosing device 2, and administration needle 4, but also power supply 5. In contrast to drive 3 for conveying medication, which remains in housing 10, and electrical control system 6 which likewise remains in the housing, power supply 5 for operating the medication dosing device must be periodically repowered, for example by replacing a battery or charging a rechargeable battery.

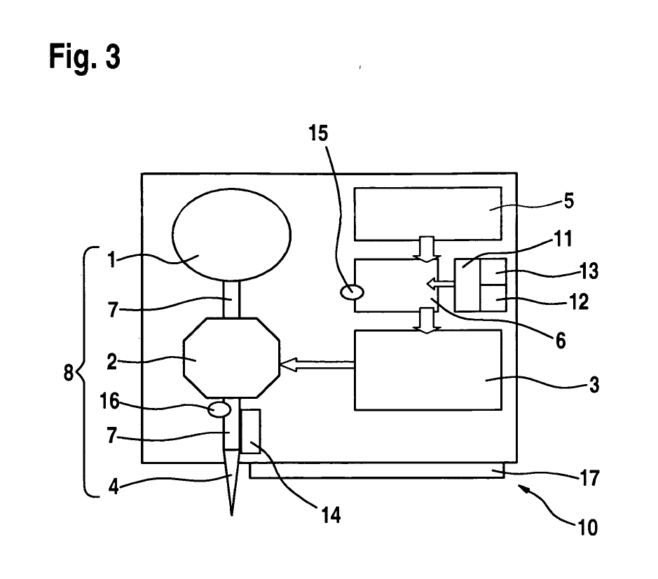

A third preferred specific embodiment is schematically illustrated in FIG. 3. Similarly as for the specific embodiment described in FIG. 1, function blocks medication reservoir 1, medication dosing device 2, and administration needle 4 are provided for the removable unit. A value, for example temperature, pressure, flow rate, blood parameters, and/or active substance levels, may be measured with the aid of a sensor component 16 additionally integrated into the removable unit. Sensor component 16 has fluid connections 7 to medication reservoir 1, medication dosing device 2, and administration needle 4. These four function blocks also have fluid connections 7 to one another. In housing 10, separate from removable unit 8, a sensor 15 measures a value, for example the ambient temperature, body temperature, rotational speed of drive 3 for conveying medication, etc.

State and operating data of the medication dosing device may be displayed via a display device 11. In particular the data measured by sensor 15 and/or sensor component 16 may be displayed on display device 11. Display device 11 is designed as an LED display, for example. Electrical control system 6 may be adjusted via a control element 13. In addition, the data measured by sensor 15 and/or sensor component 16 may be transmitted via communication interface 12. A needle insertion device 14 guides administration needle 4 or provides assistance for more precise positioning of administration needle 4 at an injection site. The medication dosing device may be affixed to the body with the aid of a fastening device 17.

1-12. (canceled)

13. A medication dosing device for administering a liquid medication, comprising a housing, the housing including:
a medication reservoir,
a medication dosing device,
a drive for conveying the medication,
an administration needle,
a power supply unit, and
an electrical control system,
wherein the medication reservoir, the medication dosing device, and the administration needle are jointly situated in a unit which may be inserted into the housing and removed from the housing.

14. The medication dosing device according to claim 13, wherein the removable unit is a disposable article.

15. The medication dosing device according to claim 13, wherein the removable unit further contains the power supply unit.

16. The medication dosing device according to claim 13, wherein the power supply unit in the housing at least one of (a) is designed to be separate from the removable unit and (b) is designed as a rechargeable battery.

17. The medication dosing device according to claim 13, wherein at least one of the drive for conveying the medication and the electrical control system in the housing is separate from the removable unit.

18. The medication dosing device according to claim 13, wherein at least one of a display device, a communication interface, at least one control element, and a needle insertion device is situated in the housing, separate from the removable unit.

19. The medication dosing device according to claim 18, wherein at least one sensor is situated in the housing, separate from the removable unit.

20. The medication dosing device according to claim 18, wherein at least one sensor component is situated in the removable unit.

21. The medication dosing device according to claim 13, wherein the housing has a fastening device, separate from the removable unit, for affixing to the body of a patient.

22. The medication dosing device according to claim 13, wherein the removable unit has a fastening device for affixing to the body of a patient.

23. The medication dosing device according to claim 13, wherein the medication dosing device has a micro diaphragm pump made of silicon.

24. The medication dosing device according to claim 13, wherein the medication reservoir in a delivered state is filled with a medication.
